(19) United States
(12) Patent Application Publication
(10) Pub. No.: US 2023/0364129 A1
(43) Pub. Date: Nov. 16, 2023

(54) TOPICAL COMPOSITIONS

(71) Applicant: Prime Performance Research LLC, Astatula, FL (US)

(72) Inventors: Dorian FARMER, Orlando, FL (US); Marilyn LITTLE, Delray Beach, FL (US)

(21) Appl. No.: 18/196,972

(22) Filed: May 12, 2023

Related U.S. Application Data
(60) Provisional application No. 63/342,534, filed on May 16, 2022.
(60) Provisional application No. 63/412,965, filed on Oct. 4, 2022.

(51) Int. Cl. A61K 31/737; A61K 38/05; A61K 47/02; A61K 47/46; A61K 47/10; A61K 47/18; A61K 47/38

(52) U.S. Cl. CPC A61K 31/737; A61K 38/05; A61K 47/02; A61K 47/46; A61K 47/10; A61K 47/183; A61K 47/38; A61K 9/0014

(57) ABSTRACT

Provided herein are pharmaceutical compositions, such as topical compositions, comprising carnosine and chondroitin sulfate. Also provided herein are methods of preparing the pharmaceutical compositions and methods of using the same for treating inflammation and diseases associated with inflammation and/or autoimmune reactions.

### CROSS-REFERENCE TO RELATED APPLICATIONS

This application claims the benefit of priority of U.S. Provisional Application Nos. 63/412,965, filed Oct. 4, 2022, and 63/342,534, filed May 16, 2022, the content of each of which is incorporated herein by reference in its entirety for all purposes.

### BACKGROUND

### Field of the Invention

In various embodiments, the present invention generally relates to novel chemical compounds, methods of making the same and to pharmaceutical preparations in which one or more of said compounds is an active ingredient. In some embodiments, the present disclosure relates to a composition for topical use, such as a topical gel-cream having the following products: aloe vera, magnesium sulfate, phenoxyethanol, tetrasodium glutamate diacetate, L-carnosine, Chondroitin 4 sulfate, and hydroxyethyl cellulose.

### Background Art

A. Carnosine (beta-alanyl-L-histidine) is a dipeptide molecule, made up of the amino acids beta-alanine and histidine. Carnosine is synthesized in the body from beta-alanine and histidine and the most abundant source is in skeletal muscle. The molecular structure of carnosine is the following:

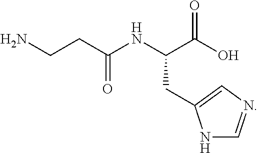 [SMILES: NCCC(=O)N[C@@H](Cc1cnc[nH]1)C(=O)O]

Carnosine physiological properties and actions include:
Neutral molecule with a positive and negative end; called a zwitterion (Boldyrev, 2013).
Polydentate ligand having several binding sites: two imidazole nitrogen atoms, one carboxylic acid group and one amino group.
Metal-ion chelation; forms complexes with metals of the first transition metal series such as Cu²⁺, Co²⁺, Ni²⁺, Cd²⁺, and Zn²⁺.
Antioxidant capacity; scavenges reactive oxygen species (ROS).
pH buffering
Overcome muscle fatigue:
High-intensity exercise leads to reductions in muscle substrates (ATP, PCr, and glycogen) and a subsequent accumulation of metabolites (ADP, Pi, H⁺, and Mg²⁺) with a possible increase in free radical production. These factors independently and collectively have deleterious effects on muscle, with significant repercussions on high-intensity performance or training sessions. The effect of carnosine on overcoming muscle fatigue appears to be related to its ability to buffer the increased H⁺ concentration following high-intensity work. (Begum, 2005).
Anti-tumor activity
L-carnosine induces cell cycle arrest and apoptosis in human colorectal cancer cells by suppressing of NF-κB/STAT1 signaling (Lee, 2018)

B. Chondroitin-4-sulfate (C4S)

Chondroitin sulfate is a sulfated glycosaminoglycan (GAG) composed of a chain of alternating sugars (N-acetylgalactosamine and glucuronic acid). It is usually found attached to proteins as part of a proteoglycan. Chondroitin sulfate A or its systemic name Chondroitin-4-sulfate (C4S, also referred to herein as chondroitin 4-sulfate or chondroitin 4 sulfate) has the sulfate attached to carbon 4.

The effect of chondroitin sulfate in people with osteoarthritis is likely the result of a number of reactions including its anti-inflammatory activity, the stimulation of the synthesis of proteoglycans and hyaluronic acid, and the decrease in catabolic activity of chondrocytes, inhibiting the synthesis of proteolytic enzymes, nitric oxide, and other substances that contribute to damage the cartilage matrix and cause death of articular chondrocytes. A recent review summarizes data from relevant reports describing the biochemical basis of the effect of chondroitin sulfate on osteoarthritis articular tissues (Monfort, 2008).

The molecular structure of C4S is shown below:

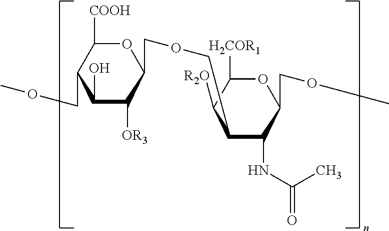

Chemical structure of one unit in a chondroitin sulfate chain. Chondroitin-4-sulfate: R₁═H; R₂═SO₃H; R₃═H. Chondroitin-6-sulfate: R₁═SO₃H; R₂, R₃═H.

In one study, Zhang et al. determined the signaling pathway involved in immune response of CS and its N-deacetylated derivative (dCS). Their data indicated that both CS and dCS could activate the NF-κB transcription factor in antigen presenting cells and induce TNF-α production through the TLR/MyD88 pathway (Zhang, 2015).

An electron microscopic picture of liposomal C4S is shown in FIG. 1 herein.

A liposome is a spherical vesicle having at least one lipid bilayer. See FIG. 2. The liposome can be used as a drug delivery vehicle for administration of nutrients and pharmaceutical drugs, such as lipid nanoparticles in mRNA vaccines, and DNA vaccines. Liposomes can be prepared by disrupting biological membranes, such as by sonication (Cevc, 1993).

A liposome has an aqueous solution core surrounded by a hydrophobic membrane, in the form of a lipid bilayer; hydrophilic solutes dissolved in the core cannot readily pass through the bilayer. Hydrophobic chemicals associate with the bilayer. A liposome can be hence loaded with hydrophobic and/or hydrophilic molecules. To deliver the molecules to a site of action, the lipid bilayer can fuse with other bilayers such as the cell membrane, thus delivering the liposome contents; this is a complex and non-spontaneous event, however, that does not apply to nutrients and drug delivery (Cevc, 1993). Clinical applications:

Inflammatory Disease of the Joints:

Inflammatory reactions are primarily originated by infectious agents, immune reactions and by sterile tissue lesions that activate membrane receptors by means of pathogen-associated molecular patterns, tissue breakdown products and cytokines. The activation of membrane receptors triggers the phosphorylation of mitogen activated protein kinases and of the nuclear factor kappaB (NF-kappaB). The binding of NF-kappaB to the promoter of target genes enhances the expression of pro-inflammatory cytokines, inducible nitric oxide synthase, cyclooxygenase 2, phospholipase A2, and matrix metalloproteases, proteins that contribute to tissue damage and to the inflammatory reaction. The activation of NF-kappaB has a key role in the immune homeostasis and the inflammatory response and therefore, in the pathogenesis of numerous diseases (Vallieres, 2010).

Carnosine Application:

Hyaluronan (hyaluronic acid) is a high-molecular-mass polysaccharide found in the extracellular matrix, especially of soft connective tissues. Hyaluronan is similar to the fluid that surrounds the joints in your body. This fluid acts as a lubricant and shock absorber for the joints. Hyaluronan is used to treat knee pain caused by osteoarthritis. Hyaluronan is usually given after other arthritis medications have been tried without success.

In a reaction system with the prevalence of hydroxyl and/or peroxy-type radicals, carnosine in 200 μmol/L concentration tested exerted a protective action on hyaluronan degradation (Drafi, 2010).

The causative factors of joint inflammation and disease are complex and as an example, oxygen metabolism plays an important role in the pathogenesis of rheumatoid arthritis (RA). Reactive oxygen species (ROS) produced in excessive amounts under some pathological states exceed the physiological ROS buffering capacity and result in oxidative stress (OS). Excessive production of ROS can damage proteins, lipids, nucleic acids, and matrix components, key agents in maintaining healthy joints (Bauerova, 1999; Chuand, 2014).

Preclinical experiments showed that carnosine may have a systemic anti-inflammatory effect as well as protecting against cartilage degradation in “arthritic joints.” (Ponist, 2016). It is an accepted fact that in development of rheumatoid arthritis IL-1 is one of the pivotal cytokines in initiating the disease. Beneficial effects of carnosine are manifested in reduction of systemic oxidative stress and reduced level of IL-1a in plasma (Bauerova, 2010).

C4S Application:

Chondroitin sulfate (CS) is able to diminish NF-kappaB activation and nuclear translocation in chondrocytes and synovial membrane, effects that may explain the benefits of CS in osteoarthritis. In addition, systemic CS reduces NF-kappaB nuclear translocation in macrophages and hepatocytes, raising the hypothesis that CS might be of benefit to treat other diseases with a strong inflammatory component. Chondroitin sulfate (CS) is able to diminish NF-kappaB activation and nuclear translocation in chondrocytes and synovial membrane, effects that may explain the benefits of CS in osteoarthritis. In addition, systemic CS reduces NF-kappaB nuclear translocation in macrophages and hepatocytes, raising the hypothesis that CS might be of benefit to treat other diseases with a strong inflammatory component (Vallieres, 2010).

Several animal studies have demonstrated that orally administered CS significantly inhibited hind paw oedema, synovitis and destruction of the articular cartilage in a dose-dependent manner. CS may reduce inflammatory processes by acting on the nuclear translocation of NF-κB, which is closely associated with the blood biomarkers of inflammation, primarily IL-1, IL-6 and C-reactive protein (Volpi, 2011).

C. Aloe Vera:

Produced from a succulent plant species of the hyenus aloe Aloe gel is used to make a topical medication.

D. Magnesium Sulfate (MgSO4) Heptahydrate Powder:

The presence of magnesium ions (Mg2+) in combination with carnosine has been found to increase levels of carnosine in the skin. Magnesium is highly effective in breaking down oil in skin pores, restoring the skin's oil balance, clearing and opening up pores.

E. Phenoxyethanol:

Phenoxyethanol is the organic compound with the formula C₆H₅OC₂H₄OH. It is a colorless oily liquid and is used as a preservative.

F. Tetrasodium Glutamate Diacetate:

Tetrasodium Glutamate Diacetate is a preservative booster made from plant material. Its chief component is L-glutamic acid, an amino acid used by almost all living beings in the biosynthesis of proteins.

F. Hydroxyethyl Cellulose:

Hydroxyethyl cellulose is a gelling and thickening agent derived from cellulose.

### BRIEF SUMMARY

In various embodiments, the present disclosure generally provides a pharmaceutical composition, such as a topical composition, comprising carnosine and chondroitin sulfate, in particular, L-carnosine and chondroitin 4-sulfate. The carnosine and chondroitin sulfate are typically included in a topical vehicle, such as water. The carnosine and chondroitin sulfate are typically included in an amount that is physiologically active, for example, for reducing inflammation. In some embodiments, the topical composition can include one or more ingredients selected from a lubricant, a thickening agent, a carrier, a preservative, a surfactant, moisturizing agents, and emollients. In some embodiments, the topical composition can further include one or more, e.g., all, ingredients selected from aloe vera, magnesium sulfate, phenoxyethanol, tetrasodium glutamate diacetate, and hydroxyethyl cellulose. Useful ingredients and amounts thereof for the composition herein include any of those described herein.

In some specific embodiments, the present invention provides a topical composition (topical formulation) of aloe vera, magnesium sulfate, phenoxyethanol, tetrasodium glutamate diacetate, L-carnosine, Chondroitin 4 sulfate, and hydroxyethyl cellulose. The topical composition may be in the form of a cream, gel, a solution, a lotion, a paste, an ointment, a patch with or without a reservoir, a spray, or a foam. In the present invention, the preference is a gel. The formulation may contain additives such as a lubricant, a thickening agent, a carrier, a preservative, a surfactant, moisturizing agents, and emollients. A main component to the present disclosure is the formulation containing physiologically active amounts of L-carnosine and Chondroitin 4 sulfate (C4S). The C4S product for the compositions herein is preferably a liposomal formulation.

The combination of L-carnosine and C4S may be present in the formulation in concentrations ranging from about 0.5 to 15 wt %, such as about 0.5-5 wt %, about 5-10 wt %, or about 5-15 wt %, of the topical formulation, which represents a physiologically active amount of this active formulation.

Some embodiments of the present disclosure are directed to a method of preparing a topical composition comprising carnosine and chondroitin sulfate, in particular, L-carnosine and chondroitin 4-sulfate, a topical vehicle, and optional one or more other ingredients. In some embodiments, the method comprises mixing the L-carnosine and chondroitin 4 sulfate, and optional one or more other ingredients, in the topical vehicle, such as water, to form a homogenous mixture. The combined amount of L-carnosine and chondroitin 4 sulfate for the topical composition typically ranges from about 5-15 wt %, preferably, about 5-10 wt % of the topical composition.

In some embodiments, the topical composition further comprises a magnesium salt and the method comprises 1) mixing a magnesium salt described herein with L-carnosine in the topical vehicle, such as water, to form a magnesium carnosine solution; and then 2) mixing the magnesium carnosine solution with chondroitin 4 sulfate, and remaining ingredient(s), to form the topical composition. The ingredients and amounts thereof useful for the topical composition include any of those described herein.

Some embodiments of the present disclosure are directed to the use of the topical compositions described herein. For example, in some embodiments, the present disclosure provides a method of treating inflammation. In some embodiments, the method comprises topically applying an effective amount of the topical composition described herein to a subject in need thereof. In some embodiments, the inflammation is within a joint of the subject at a synovial membrane and/or within a joint cartilage.

In some embodiments, the present disclosure provides a method of treating a disease associated with an inflammatory and/or autoimmune reaction. For example, in some embodiments, the method comprises topically applying an effective amount of the topical composition described herein to a subject in need thereof. Diseases that can be treated with the methods herein include any of those described herein, for example, osteoarthritis.

Exemplary embodiments of the present disclosure are also shown in the following enumerated Embodiments 1-32:
Embodiment 1. A topical composition comprising L-carnosine, chondroitin 4 sulfate, and a topical vehicle, wherein a combined amount of L-carnosine and chondroitin 4 sulfate ranges from about 0.5-15 wt % of the topical composition, preferably, the chondroitin 4 sulfate is substantially pure, e.g., as described herein.
Embodiment 2. The topical composition of Embodiment 1, wherein the combined amount of L-carnosine and chondroitin 4 sulfate ranges from about 5-10 wt % of the topical composition.
Embodiment 3. The topical composition of Embodiment 1 or 2, wherein a weight ratio of L-carnosine to chondroitin 4 sulfate ranges from about 1:1 to about 10:1.
Embodiment 4. The topical composition of Embodiment 3, wherein the weight ratio of L-carnosine to chondroitin 4 sulfate ranges from about 2:1 to about 5:1, such as about 3:1 or about 4:1.
Embodiment 5. The topical composition of any of Embodiments 1-4, wherein the chondroitin 4 sulfate is in the form of a liposomal chondroitin 4 sulfate.
Embodiment 6. The topical composition of any of Embodiments 1-5, wherein the combination of L-carnosine and chondroitin 4 sulfate achieves a synergistic antioxidant and/or anti-inflammatory effect.
Embodiment 7. The topical composition of any of Embodiments 1-6, further comprising a magnesium salt, preferably, a combined amount of magnesium and L-carnosine is in the range of about 5-10%, for example, about 5%, 5.5%, 6%, 6.5%, 7%, 8%, 9%, 10%, or any range or value between the recited values, such as about 5-6%, by weight of the topical composition.
Embodiment 8. The topical composition of Embodiment 7, wherein a molar ratio of magnesium to L-carnosine in the topical composition is about 1:1.
Embodiment 9. The topical composition of any of Embodiments 1-8, further comprising one or more ingredients selected from a further anti-inflammatory agent, a preservative, a gel forming agent, a moisturizing agent, a carrier, a surfactant, a chelating agent, and an emollient.
Embodiment 10. The topical composition of any of Embodiments 1-9, further comprising one or more (e.g., all) ingredients selected from aloe vera, phenoxyethanol, tetrasodium glutamate diacetate, and hydroxyethyl cellulose.
Embodiment 11. The topical composition of any of Embodiments 1-10, wherein the topical vehicle is water.
Embodiment 12. The topical composition of any of Embodiments 1-11, in the form of a cream, gel, a solution, a lotion, a paste, an ointment, a patch with or without a reservoir, a spray, or a foam.
Embodiment 13. The topical composition of any of Embodiments 1-11, in the form of a gel.
Embodiment 14. The topical composition of any of Embodiments 1-13, comprising the following ingredients:
   1) aloe vera, in an amount of about 0.5-5% by weight of the topical composition;
   2) a magnesium salt, in an amount of about 0.5-10% by weight of the topical composition;
   3) phenoxyethanol, in an amount of about 0.5-5% by weight of the topical composition;
   4) tetrasodium glutamate diacetate, in an amount of about 0.1-5% by weight of the topical composition;
   5) L-carnosine, in an amount of about 0.5-10% by weight of the topical composition;
   6) hydroxyethyl cellulose, in an amount of about 0.5-5% by weight of the topical composition;
   7) liposomal chondroitin 4 sulfate, in an amount of about 0.1-5% by weight of the topical composition; and
   8) the topical vehicle.
Embodiment 15. The topical composition of Embodiment 14, characterized by one or more (e.g., all) of the following:
   1) the aloe vera is in an amount of about 2-4% by weight of the topical composition;
   2) the magnesium salt is a magnesium sulfate, which is in an amount of about 1-3%, about 4-6%, or about 5-10% by weight of the topical composition;
   3) the phenoxyethanol is in an amount of about 1-3% by weight of the topical composition;
   4) the tetrasodium glutamate diacetate is in an amount of about 0.1-1% or about 1-3% by weight of the topical composition;
   5) the L-carnosine is in an amount of about 1-4%, about 1.5-2.5%, about 4-6%, or about 5-10% by weight of the topical composition;
   6) the hydroxyethyl cellulose is in an amount of about 1-3% by weight of the topical composition;
   7) the liposomal chondroitin 4 sulfate is in an amount of about 0.5-2% or about 1-3% by weight of the topical composition; and
   8) the topical vehicle is water.
Embodiment 16. The topical composition of Embodiment 14, characterized by one or more (e.g., all) of the following:
   1) the aloe vera is in an amount of about 3.1% by weight of the topical composition;
   2) the magnesium salt is a magnesium sulfate, which is in an amount of about 3% or about 5% by weight of the topical composition;
   3) the phenoxyethanol is in an amount of about 1.7% by weight of the topical composition;
   4) the tetrasodium glutamate diacetate is in an amount of about 0.5% or about 1.7% by weight of the topical composition;
   5) the L-carnosine is in an amount of about 3%, or about 5% by weight of the topical composition;
   6) the hydroxyethyl cellulose is in an amount of about 2% or about 2.5% by weight of the topical composition;
   7) the liposomal chondroitin 4 sulfate is in an amount of about 1.2% by weight of the topical composition; and
   8) the topical vehicle is water.
Embodiment 17. The topical composition of any of Embodiments 14-16, wherein the combined amount of magnesium and L-carnosine ranges from about 5-10%, preferably, about 5-6% by weight of the topical composition.
Embodiment 18. The topical composition of Embodiment 14, comprising, for every 32 oz of the composition, about 1 oz aloe vera (liquid form), about 48.05 grams magnesium sulfate heptahydrate, about 15 grams of phenoxyethanol, about 15 grams of tetrasodium glutamate diacetate, about 45 grams of L-carnosine, about 23 grams of hydroxyethyl cellulose, about 18 grams of liposomal chondroitin 4 sulfate (containing about 60 or 70 weight % chondroitin 4 sulfate) and water Q.S.
Embodiment 19. A method of preparing a topical composition comprising L-carnosine, chondroitin 4 sulfate, a topical vehicle, and optional one or more other ingredients, the method comprising mixing the L-carnosine, chondroitin 4 sulfate, and optional one or more other ingredients, in the topical vehicle to form a homogenous mixture, wherein a combined amount of L-carnosine and chondroitin 4 sulfate ranges from about 5-15 wt %, preferably, about 5-10 wt % of the topical composition, preferably, the combination of L-carnosine and chondroitin 4 sulfate achieves a synergistic antioxidant and/or anti-inflammatory effect, preferably, the chondroitin 4 sulfate is substantially pure, e.g., as described herein.
Embodiment 20. The method of Embodiment 19, wherein the topical composition is characterized by: 1) a weight ratio of L-carnosine to chondroitin 4 sulfate ranges from about 1:1 to about 10:1, preferably, from about 2:1 to about 5:1, such as about 3:1 or about 4:1; and/or 2) the chondroitin 4 sulfate is in the form of a liposomal chondroitin 4 sulfate.
Embodiment 21. The method of Embodiment 19 or 20, wherein the topical composition comprises the optional one or more other ingredients, which include one or more (e.g., all) ingredients selected from aloe vera, a magnesium salt, phenoxyethanol, tetrasodium glutamate diacetate, and hydroxyethyl cellulose.
Embodiment 22. The method of Embodiment 19 or 20, comprising mixing the following ingredients in the topical vehicle:
   1) aloe vera, in an amount of about 0.5-5% by weight of the topical composition;
   2) a magnesium salt, in an amount of about 0.5-10% by weight of the topical composition;
   3) phenoxyethanol, in an amount of about 0.5-5% by weight of the topical composition;
   4) tetrasodium glutamate diacetate, in an amount of about 0.1-5% by weight of the topical composition;
   5) L-carnosine, in an amount of about 0.5-10% by weight of the topical composition;
   6) hydroxyethyl cellulose, in an amount of about 0.5-5% by weight of the topical composition; and
   7) liposomal chondroitin 4 sulfate, in an amount of about 0.1-5% by weight of the topical composition.
Embodiment 23. The method of Embodiment 19 or 20, comprising mixing the following ingredients in the topical vehicle:
   1) aloe vera, in an amount of about 2-4% by weight of the topical composition;
   2) magnesium sulfate, which is in an amount of about 1-3%, about 4-6%, or about 5-10% by weight of the topical composition;
   3) phenoxyethanol, in an amount of about 1-3% by weight of the topical composition;
   4) tetrasodium glutamate diacetate, in an amount of about 0.1-1% or about 1-3% by weight of the topical composition;
   5) L-carnosine, in an amount of about 1-4%, about 1.5-2.5%, about 4-6%, or about 5-10% by weight of the topical composition;
   6) hydroxyethyl cellulose, in an amount of about 1-3% by weight of the topical composition; and
   7) liposomal chondroitin 4 sulfate, in an amount of about 0.5-2% or about 1-3% by weight of the topical composition.
Embodiment 24. The method of Embodiment 19 or 20, comprising mixing the following ingredients in the topical vehicle:
   1) aloe vera, in an amount of about 3.1% by weight of the topical composition;
   2) magnesium sulfate, in an amount of about 3% or about 5% by weight of the topical composition;
   3) phenoxyethanol, in an amount of about 1.7% by weight of the topical composition;
   4) tetrasodium glutamate diacetate, in an amount of about 0.5% or about 1.7% by weight of the topical composition;
   5) L-carnosine, in an amount of about 3% or about 5% by weight of the topical composition;
   6) hydroxyethyl cellulose, in an amount of about 2% or about 2.5% by weight of the topical composition; and
   7) liposomal chondroitin 4 sulfate, in an amount of about 1.2% by weight of the topical composition.
Embodiment 25. The method of any of Embodiments 19-24, comprising 1) mixing a magnesium salt, or the magnesium salt of any of Embodiments 21-24, with L-carnosine in the topical vehicle to form a magnesium carnosine solution; and then 2) mixing the magnesium carnosine solution with chondroitin 4 sulfate, and any remaining ingredient(s), to form the topical composition, preferably, a combined amount of magnesium and L-carnosine ranges from about 5-10%, preferably, about 5-6% by weight of the topical composition.
Embodiment 26. The method of Embodiment 25, wherein the topical composition is characterized by a molar ratio of the magnesium salt and L-carnosine of about 1:1.
Embodiment 27. The method of any of Embodiments 19-26, wherein the topical vehicle is water.
Embodiment 28. The method of any of Embodiments 19-27, wherein the topical composition is in the form of a gel.
Embodiment 29. The topical composition produced by the method of any of Embodiments 19-28.
Embodiment 30. A method of treating inflammation, comprising topically applying an effective amount of the topical composition of any of Embodiments 1-18 and 29 to a subject in need thereof.
Embodiment 31. The method of Embodiment 30, wherein the inflammation is within a joint of the subject at a synovial membrane and/or within a joint cartilage.
Embodiment 32. A method of treating a disease associated with an inflammatory and/or autoimmune reaction, comprising topically applying an effective amount of the topical composition of any of Embodiments 1-18 and 29 to a subject in need thereof.

It is to be understood that both the foregoing summary and the following detailed description are exemplary and explanatory only, and are not restrictive of the invention herein.

### BRIEF DESCRIPTION OF THE FIGURES

FIG. 1 shows an electron microscopic picture of liposomal C4S.

FIG. 2 shows a typical structure of a liposome.

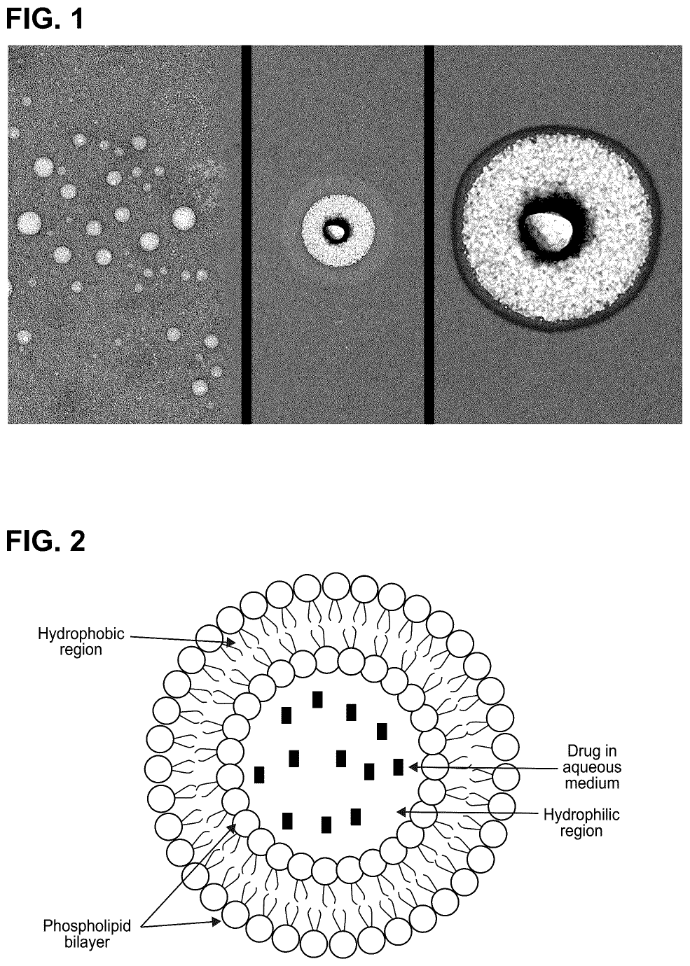

### DETAILED DESCRIPTION

The present disclosure generally relates to pharmaceutical compositions comprising carnosine and chondroitin sulfate, in particular, L-carnosine and chondroitin 4-sulfate, methods of preparation and methods of using the same. The present disclosure is based in part on the discovery that a stable active topical composition can be prepared based on L-carnosine and chondroitin 4-sulfate, which can be advantageously used as antioxidants and/or to treat inflammation, such as a local inflammation, for example, inflammation within joints at the synovial membrane and/or within the joint cartilage, with or without relying on a systemic effect. In addition, the combination of L-carnosine and chondroitin 4-sulfate in the topical composition herein can provide a synergistic effect, such as a synergistic antioxidant and/or anti-inflammatory effect. Without wishing to be bound by theories, it is believed that carnosine and C4S synergistically work together in their unique abilities to be both antioxidants (e.g., neutralizing reactive oxygen species or free radicals) and deglycating agents, as well as reacting with Cu²⁺ or Fe²⁺ which are elevated in inflammatory oxidative stress conditions. Both carnosine and C4S uniquely multitask as an antioxidant and a deglycator. The physiologic antioxidant actions of carnosine and C4S are complementary to each other, and can be synergistically combined. The topical compositions described herein are useful for treating various inflammatory and/or autoimmune related conditions, including any of those described herein. While many of the embodiments described herein are directed to topical compositions or topical uses, pharmaceutical compositions comprising L-carnosine and chondroitin 4-sulfate, in amounts described herein, formulated for other routes of administration, such as for oral administration or injections, and uses thereof, are also novel compositions and methods of the present disclosure. In some embodiments, the present disclosure also provides compositions comprising L-carnosine and chondroitin 4-sulfate useful as a supplement for nutraceutical applications.

### Topical Compositions

In some embodiments, the present invention provides a topical composition (alternatively referred to herein as topical formulation) comprising L-carnosine, chondroitin 4 sulfate, and a topical vehicle. Topical compositions as used herein generally refer to any composition that can be applied to a subject (e.g., a human subject) topically, regardless of the location of the effect, and topical administration should be understood similarly. In some embodiments, the topical composition is for achieving a localized effect. The topical compositions herein are typically pharmaceutically acceptable and can be used for topical administration to a human subject.

The amount of L-carnosine and chondroitin 4-sulfate is not particularly limited and includes any of those described herein. Typically, the combined amount of L-carnosine and chondroitin 4-sulfate in the topical composition is in a physiologically active amount. In some embodiments, the combined amount of L-carnosine and chondroitin 4 sulfate ranges from about 0.5-15 wt % of the topical composition, such as about 0.5%, 1%, 2%, 3%, 4%, 5%, 6%, 7%, 8%, 9%, 10%, 12%, 15%, or any range or value between the recited values, such as about 5-10%, by weight of the topical composition. More preferably, the combined amount of L-carnosine and chondroitin 4 sulfate ranges from about 5-10 wt % of the topical composition. In some embodiments, the combined amount of L-carnosine and chondroitin 4 sulfate is such that topically applying the topical composition to a subject can reduce inflammation in the subject. The weight percentages (e.g., wt % or weight %) described herein, unless otherwise specified or contrary from context, should be understood as referring to the weight percentages based on the final topical composition.

In some embodiments, the L-carnosine is included in the topical composition in an amount of about 0.5-10% (e.g., about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, or any range or value between the recited values, such as about 1-4%, about 1.5-2.5%, about 4-6%, or about 5-10%) by weight of the topical composition.

In some embodiments, the chondroitin 4-sulfate is included in the topical composition in an amount of about 0.1-5% (e.g., about 0.1%, 0.5%, 1%, 1.5%, 2%, 2.5%, 3%, 3.5%, 4%, 4.5%, 5%, or any range or value between the recited values, such as about 0.5-2% or about 1-3%) by weight of the topical composition.

In embodiments, the topical composition can also be characterized by a weight ratio of L-carnosine to chondroitin 4 sulfate (i.e., L-carnosine/C4S) ranging from about 1:10 to about 10:1. In some embodiments, the weight ratio of L-carnosine to chondroitin 4 sulfate can be about 1:5, 1:2, 1:1, 2:1, 3:1, 4:1, 5:1, 6:1, 7:1, 8:1, 9:1, 10:1, or any range or value between the recited values. In some embodiments, the weight ratio of L-carnosine to chondroitin 4 sulfate in the topical composition herein ranges from about 1:1 to about 10:1. In some preferred embodiments, the weight ratio of L-carnosine to chondroitin 4 sulfate in the topical composition herein ranges from about 2:1 to about 5:1, such as about 3:1 or about 4:1.

The L-carnosine suitable for use in the topical compositions herein is not particularly limited, which can be in a free form (i.e., no external acid or base addition) or in a pharmaceutically acceptable salt form (e.g., an acid addition salt or a base addition salt form). For the avoidance of doubt, unless otherwise specified or contrary from context, when a salt of L-carnosine is used, the weight percentage of L-carnosine is calculated based on the corresponding weight of the free form. In some embodiments, L-carnosine in a free form is used for preparing the topical composition herein.

The C4S useful for the present invention herein is not particularly limited as long as it is pharmaceutically acceptable and can be used for topical administration to treat a subject in need, preferably, a human subject. Suitable C4S includes any of those known in the art, including those commercially available. Typically, chondroitin sulfate can be extracted from animal or marine sources and purified. The average molecular weight (number or weight based average molecular weight) of the chondroitin sulfate can vary, e.g., from about 50,000 Daltons to about 100,000 Daltons. In some embodiments, the chondroitin 4 sulfate included in the topical formulation herein has a number or weight based average molecular weight ranging from about 10,000 Daltons to about 50,000 Daltons. In some embodiments, the chondroitin 4 sulfate included in the topical formulation herein has a number or weight based average molecular weight ranging from about 1,000 Daltons to about 10,000 Daltons, such as about 2,000 to about 6,000 Daltons, about 1,000 to about 5,000 Daltons, about 3,000 to about 7,000 Daltons, about 5,500 Daltons, etc. The C4S is typically used in a salt form, such as in a sodium salt. The calculation of weight percentages herein is based on the weight of the C4S in the form used, for example, when sodium C4S is used, the weight percentage is calculated based on the weight of sodium C4S.

In some preferred embodiments, the chondroitin 4 sulfate for the topical compositions herein is substantially pure. For example, in some embodiments, the chondroitin 4-sulfate has a purity by weight (on a dry basis) of greater than 80%, preferably, greater than 90%, greater than 95%, or greater than 98%. The chondroitin 4-sulfate is typically free of pyrogens, i.e., no pyrogens is detected in the product. In some preferred embodiments, the chondroitin 4 sulfate can be characterized by one or more (such as all) of the following characteristics: 1) not more than 2% of any individual impurity (such as chondroitin 6-sulfate); 2) a 5% solution shows a specific optical rotation of between −20° to −30°; 3) a 5% solution shows a pH ranging from 5.5-7.5; and 4) an intrinsic viscosity of about 0.01 to 0.15 m³/kg. In some embodiments, the chondroitin 4 sulfate is freely soluble in water. In some embodiments, the chondroitin 4 sulfate is in an injectable grade. In some embodiments, the chondroitin 4 sulfate is derived from bovine trachea. In some preferred embodiments, the C4S has a number or weight based average molecular weight ranging from about 1,000 Daltons to about 10,000 Daltons, such as about 2,000 to about 6,000 Daltons, about 1,000 to about 5,000 Daltons, about 3,000 to about 7,000 Daltons, about 5,500 Daltons, etc.

In some preferred embodiments, the chondroitin 4 sulfate for the topical compositions herein is encapsulated in a liposome, alternatively referred to herein as liposomal C4S. Liposomal C4S suitable for use for the present invention is not particularly limited and include any of those known or commercially available liposomal C4S products. In some embodiments, the C4S used for the liposomal C4S is a substantially pure C4S described herein. In some embodiments, the C4S is a low molecular weight C4S, such as those having a number or weight based average molecular weight ranging from about 1,000 Daltons to about 10,000 Daltons, such as about 2,000 to about 6,000 Daltons, about 1,000 to about 5,000 Daltons, about 3,000 to about 7,000 Daltons, about 5,500 Daltons, etc. The particle size of the liposomal C4S is not particularly limited. For example, in some embodiments, the liposomal C4S can be characterized as having an average particle size of less than 5 μm, preferably, less than 1 μm, such as less than 0.5 μm, as measured by DLS. Liposomal C4S products can be typically prepared from positively charged liposomes, which can be used to encapsulate C4S under suitable conditions. Examples of preparing liposomal C4S can be found at Craciunescu O. et al. Studia Universitatis “Vasile Goldş”, Seria Ştiinţele Vieţii 24:93-101 (2014), which details a preparation of a liposomal C4S using C4S sodium salt from bovine trachea and a liposome system of PC:DOPE:Chol:SA, in 4:2:3:1 molar ratio, with a loading or encapsulation efficiency of as high as 68.7%. (PC represents L-α-phosphatidylcholine Type XVI-E from egg yolk; DOPE represents dioleoyl-phosphatidyl-ethanolamine; SA represents stearyl amine; and Chol represents cholesterol.) Suitable liposomal C4S also include those commercially available, for example, the product sold under the name PureC4S. Typically, the liposomal C4S can have about 40-70% by weight of C4S. For example, in some embodiments, the liposomal C4S can be characterized as having about 60% by weight of C4S, with a ratio of C4S to the lipid blend (liposomal vehicle) of about 1.5:1. In some embodiments, the liposomal C4S can be characterized as having about 70% by weight of C4S, with a ratio of C4S to the lipid blend (liposomal vehicle) of about 2.33:1.

In some embodiments, the topical composition further comprises a magnesium salt. In some embodiments, the magnesium salt is present in a concentration such that the molar ratio of magnesium to L-carnosine in the topical composition is about 1:1. As discussed herein, in some embodiments, the magnesium salt can be mixed with L-carnosine in the topical vehicle first to form a magnesium L-carnosine solution, which is then mixed with remaining ingredient(s). In some embodiments, the magnesium salt can also be mixed with L-carnosine and remaining ingredient(s) in the topical vehicle without any particular order.

The amount of the magnesium salt is typically about 0.5-10% (e.g., about 0.5%, 1%, 2%, 3%, 4%, 5%, 6%, 7%, 8%, 9%, 10%, or any range or value between the recited values, such as about 1-3%, about 4-6%, or about 5-10%) by weight of the topical composition. In one embodiment, the combined amount of Magnesium and L-Carnosine in the formulation is in the range of about 5-10 wt %, for example, about 5%, 5.5%, 6%, 6.5%, 7%, 8%, 9%, 10%, by weight, or any range or value between the recited values, such as about 5-6 wt %, of the topical formulation. The amount of Magnesium can be calculated based on the amount of Magnesium salt, such as Magnesium Sulfate heptahydrate, used in preparing the topical formulation. For example, 48.05 grams of Magnesium Sulfate heptahydrate contain about 4.74 grams of Magnesium. In some embodiments, the molar ratio of Magnesium to L-Carnosine in the topical formulation is about 1:1.

In some embodiments, the topical composition can further comprise one or more additional ingredients selected from a further anti-inflammatory agent, a preservative, a gel forming agent, a moisturizing agent, a carrier, a surfactant, a chelating agent, an emollient, etc. For example, in some embodiments, the topical composition can further comprise one or more ingredients selected from aloe vera, phenoxyethanol, tetrasodium glutamate diacetate, and hydroxyethyl cellulose. In some embodiments, the topical composition can comprise aloe vera, phenoxyethanol, tetrasodium glutamate diacetate, and hydroxyethyl cellulose. The amounts of the additional ingredients are not particularly limited. In some embodiments, an aloe vera (preferably, a liquid form) is included in the topical composition, which can be typically present in an amount of about 0.5-5% (e.g., about 0.5%, 1%, 2%, 3%, 4%, 5%, or any range or value between the recited values, such as about 2-4%) by weight of the topical composition. In some embodiments, phenoxyethanol is included in the topical composition, which can be typically present in an amount of about 0.5-5% (e.g., about 0.5%, 1%, 2%, 3%, 4%, 5%, or any range or value between the recited values, such as about 1-3%) by weight of the topical composition. In some embodiments, tetrasodium glutamate diacetate is included in the topical composition, which can be typically present in an amount of about 0.1-5% (e.g., about 0.1%, 0.5%, 1%, 2%, 3%, 4%, 5%, or any range or value between the recited values, such as about 0.1-1% or about 1-3%) by weight of the topical composition. In some embodiments, hydroxyethyl cellulose is included in the topical composition, which can be typically present in an amount of about 0.5-5% (e.g., about 0.5%, 1%, 2%, 3%, 4%, 5%, or any range or value between the recited values, such as about 1-3%) by weight of the topical composition.

In some exemplary embodiments, the topical composition can comprise (1) L-carnosine; (2) chondroitin 4 sulfate; (3) a magnesium salt; (4) a further anti-inflammatory agent; (5) a preservative; (6) a gel-forming agent; and (7) the topical vehicle. In some embodiments, the topical composition comprises (1) L-carnosine; (2) chondroitin 4 sulfate; (3) a magnesium salt; (4) aloe vera; (5) a preservative; (6) a gel-forming agent; and (7) the topical vehicle. In some embodiments, the topical composition comprises (1) L-carnosine; (2) chondroitin 4 sulfate; (3) a magnesium salt; (4) aloe vera; (5) phenoxyethanol; (6) a gel-forming agent; and (7) the topical vehicle. In some embodiments, the topical composition comprises (1) L-carnosine; (2) chondroitin 4 sulfate; (3) a magnesium salt; (4) aloe vera; (5) phenoxyethanol; (6) hydroxyethyl cellulose; and (7) the topical vehicle. In some embodiments, the topical composition can comprise a chelating agent. For example, in some embodiments, the topical composition comprises (1) L-carnosine; (2) chondroitin 4 sulfate; (3) a magnesium salt; (4) aloe vera; (5) phenoxyethanol; (6) hydroxyethyl cellulose; (7) tetrasodium glutamate diacetate; and (8) the topical vehicle. Useful amounts and/or identities of these ingredients include any of those described herein in any combinations.

Typically, the topical vehicle herein is water.

The topical composition herein can be in any suitable form. For example, in some embodiments, the topical composition is in the form of a cream, gel, a solution, a lotion, a paste, an ointment, a patch with or without a reservoir, a spray, or a foam. In some preferred embodiments, the topical composition herein is in the form of a gel, more preferably, an aqueous gel.

The combinations of ingredients and amounts thereof for the topical compositions herein are not particularly limited. For example, in some specific embodiments, the topical composition comprises the following ingredients:
1) aloe vera, in an amount of about 0.5-5% by weight of the topical composition;
2) a magnesium salt, in an amount of about 0.5-10% by weight of the topical composition;
3) phenoxyethanol, in an amount of about 0.5-5% by weight of the topical composition;
4) tetrasodium glutamate diacetate, in an amount of about 0.1-5% by weight of the topical composition;
5) L-carnosine, in an amount of about 0.5-10% by weight of the topical composition;
6) hydroxyethyl cellulose, in an amount of about 0.5-5% by weight of the topical composition;
7) liposomal chondroitin 4 sulfate, in an amount of about 0.1-5% by weight of the topical composition; and
8) the topical vehicle. In some embodiments, the combined amount of L-carnosine and chondroitin 4 sulfate is in the range of about 5-10% weight of the topical composition. In some embodiments, the weight ratio of L-carnosine to chondroitin 4 sulfate ranges from about 2:1 to 5:1, such as about 3:1 or about 4:1. In some embodiments, the combined amount of magnesium and L-carnosine ranges from about 5-10%, preferably, about 5-6% by weight of the topical composition, with the molar ratio of magnesium to L-carnosine of about 1:1. In some embodiments, the topical vehicle is water.

In some specific embodiments, the topical composition comprises the following ingredients:
1) aloe vera in an amount of about 2-4% by weight of the topical composition;
2) a magnesium sulfate, which is in an amount of about 1-3%, about 4-6%, or about 5-10% by weight of the topical composition;
3) phenoxyethanol in an amount of about 1-3% by weight of the topical composition;
4) tetrasodium glutamate diacetate in an amount of about 0.1-1% or about 1-3% by weight of the topical composition;
5) L-carnosine in an amount of about 1-4%, about 1.5-2.5%, about 4-6%, or about 5-10% by weight of the topical composition;
6) hydroxyethyl cellulose in an amount of about 1-3% by weight of the topical composition;
7) liposomal chondroitin 4 sulfate in an amount of about 0.5-2% or about 1-3% by weight of the topical composition; and
8) the topical vehicle, water.

In some embodiments, the combined amount of L-carnosine and chondroitin 4 sulfate is in the range of about 5-10% weight of the topical composition. In some embodiments, the weight ratio of L-carnosine to chondroitin 4 sulfate ranges from about 2:1 to about 5:1, such as about 3:1 or about 4:1. In some embodiments, the combined amount of magnesium and L-carnosine ranges from about 5-10%, preferably, about 5-6% by weight of the topical composition, with the molar ratio of magnesium to L-carnosine of about 1:1. In some embodiments, the topical vehicle is water.

In some specific embodiments, the topical composition above can be characterized by one or more (e.g., all) of the following, as applicable:
1) the aloe vera is in an amount of about 3.1% by weight of the topical composition;
2) the magnesium salt is a magnesium sulfate, which is in an amount of about 3% or about 5% by weight of the topical composition;
3) the phenoxyethanol is in an amount of about 1.7% by weight of the topical composition;
4) the tetrasodium glutamate diacetate is in an amount of about 0.5% or about 1.7% by weight of the topical composition;
5) the L-carnosine is in an amount of about 3%, or about 5% by weight of the topical composition;
6) the hydroxyethyl cellulose is in an amount of about 2% or about 2.5% by weight of the topical composition;
7) the liposomal chondroitin 4 sulfate is in an amount of about 1.2% by weight of the topical composition; and
8) the topical vehicle is water.

### Exemplary Topical Compositions

In some embodiments, the present disclosure also provides the following exemplary topical compositions.

In one embodiment, the topical composition, such as a gel, has the ingredients as shown in the table below:

| | Typical amounts | Exemplified amount |
|---|---|---|
| | (weight % of final | (weight % of final |
| Ingredients | formulation) | formulation) |
| Aloe Vera | 0.5-5% (e.g., 2-4%) | 3.1% |
| Magnesium salt, | 0.5-10% (e.g., 1-3%, | 3%, 5% |
| such as, Magnesium | 4-6%, 5-10%, etc.) | |
| Sulfate (MgSO4), | | |
| e.g., MgSO4 | | |
| heptahydrate powder | | |
| Phenoxyethanol | 0.5-5% (e.g., 1-3%) | 1.7% |
| Tetrasodium | 0.1-5% (e.g., 0.1-1%, | 0.5%, 1.7% |
| Glutamate Diacetate | 1-3%, etc.) | |
| L- Carnosine | 0.5-10% (e.g., 1-4%, | 3%, 5% |
| | 1.5-2.5%, 4-6%, 5- | |
| | 10%, etc.) | |
| Hydroxyethyl | 0.5-5% (e.g., 1-3%) | 2%, 2.5% |
| cellulose | | |
| Liposomal | 0.1-5% (e.g., 0.5-2%, | 1.2% |
| Chondroitin 4 sulfate | 1-3%, etc.) | |
| (C4S) | | |
| Topical vehicle, | Q.S. | |
| such as water | | |

The ingredients listed above are readily available. For example, Aloe Vera (liquid form) can be purchased from New Directions Aromatics (Catalog No. 16001019032). Liposomal C4S is also known, including those marketed as PureC4S. To be clear, the weight percentage of Aloe Vera herein is based on Aloe Vera in liquid form. The weight percentage of liposomal C4S herein, such as those listed above in the table, is calculated based on the weight of C4S, i.e., not counting the weight of the liposome vehicle. A liposomal C4S used for the formulation may typically have about 60 weight % or about 70 wt % of C4S. However, liposomal C4S products with a different C4S loading may also be used. The weight percentages shown in the table above, should be understood as being preceded by the word “about”. In some embodiments, the weight percentages shown in the table above refer to the stated value or any value within +/−20% of the stated value, more preferably, within +/−10% of the stated value. For example, 5 wt % shown in the table should include 5 wt % and any value within 4-6%, more preferably, 4.5-5.5 wt %.

In one embodiment, the combined amount of Magnesium and L-Carnosine in the formulation is in the range of about 5-10 wt %, such as about 5-6 wt %, of the topical formulation. The amount of Magnesium can be calculated based on the amount of Magnesium salt, such as Magnesium Sulfate heptahydrate, used in preparing the topical formulation. For example, 48.05 grams of Magnesium Sulfate heptahydrate contain about 4.74 grams of Magnesium. In some embodiments, the molar ratio of Magnesium to L-Carnosine in the topical formulation is about 1:1.

In one example, the topical formulation includes, for every 32 oz of formulation, about 1 oz Aloe Vera (liquid form), about 48.05 grams Magnesium Sulfate heptahydrate, about 15 grams of Phenoxyethanol, about 15 grams of Tetrasodium Glutamate Diacetate, about 45 grams of L-Carnosine, about 23 grams of Hydroxyethyl cellulose, about 18 grams of liposomal C4S (containing about 60 weight % C4S) and water Q.S. Although it is clear, for the avoidance of doubt, the “oz” herein refers to fluid oz.

### Preparation Methods and Uses of the Topical Compositions

The topical compositions herein can be prepared by mixing the ingredients in a suitable topical vehicle, such as water, typically to obtain a homogenous mixture.

In some embodiments, the present disclosure provides a method of preparing a topical composition comprising carnosine and chondroitin sulfate, in particular, L-carnosine and chondroitin 4-sulfate, a topical vehicle, and optional one or more other ingredients. In some embodiments, the method comprises mixing the L-carnosine and chondroitin 4 sulfate, and optional one or more other ingredients, in the topical vehicle, such as water, to form a homogenous mixture. The amount of L-carnosine and chondroitin 4 sulfate and suitable and preferred ratios thereof are described herein.

In some embodiments, the topical composition further comprises a magnesium salt, and the method comprises 1) mixing a magnesium salt described herein with L-carnosine in the topical vehicle, such as water, to form a magnesium carnosine solution; and then 2) mixing the magnesium carnosine solution with chondroitin 4 sulfate, and remaining ingredient(s), to form the topical composition. The ingredients and amounts of the topical composition include any of those described herein.

In one embodiment, the topical compositions herein can be prepared by first mixing the Magnesium salt, such as Magnesium Sulfate heptahydrate, and L-Carnosine in a suitable topical vehicle, such as water, to form a Magnesium Carnosine solution, which is then followed by mixing the Magnesium Carnosine solution with the remaining ingredients. In some embodiments, the molar ratio of the Magnesium salt to L-Carnosine is about 1:1.

The topical compositions prepared by the methods herein are also novel compositions and can be used for topical administration to a subject in need.

The topical compositions herein can be used for treating a variety of conditions described above. For example, the topical compositions herein can be applied topically to a subject in need thereof to deliver a physiologically active amounts of L-carnosine and Chondroitin 4 sulfate (C4S) for treating inflammation within joints at the synovial membrane and within the joint cartilage. The formulations herein may also be used to treat diseases with an inflammatory and/or autoimmune reaction, such as inflammatory bowel disease, psoriasis, central nervous system degenerative diseases, inflammation of the eye, atherosclerosis, as well as other autoimmune diseases.

For example, in some embodiments, the present disclosure provides a method of treating inflammation, which comprises topically applying an effective amount of the topical composition herein to a subject in need thereof, preferably, a human subject. In some embodiments, the inflammation is within a joint of the subject at a synovial membrane and/or within a joint cartilage.

In some embodiments, the present disclosure provides a method of treating a disease associated with an inflammatory and/or autoimmune reaction. For example, in some embodiments, the method comprises topically applying an effective amount of the topical composition described herein to a subject in need thereof, preferably, a human subject. Diseases that can be treated with the methods herein include any of those described herein, for example, osteoarthritis.

### Definitions

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

As used herein, unless otherwise specified, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value. In one embodiment, the term “about” means within 10% of the reported numerical value. To be clear, as used herein, a range of “about x-y” or “about x to y” means the same as the range of “about x to about y”. If a unit or another characteristic follows the range of “about x-y” or “about x to y”, it should be understood that the same unit or another characteristic applies to both about x and about y. For example, “about 0.5-15 wt %” means the same as about 0.5 wt % to about 15 wt %. Other ranges preceded by the word “about” should be understood similarly. In addition, for conciseness, in a series of numerical values, if the first numeric value is modified by the word “about”, then it should be understood that all remaining numerical values in the series are also modified by the word “about”, even if some or all of these remaining numerical values in the series are not preceded by the word “about”. For example, “about 0.5%, 1%, 2%, 3%, 4%, . . . ” means the same as “about 0.5%, about 1%, about 2%, about 3%, about 4%, . . . ”.

The term “subject” as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

### REFERENCES

Bauerova, K and Bezek, S. 1999. Role of reactive oxygen and nitrogen species in etiopathogenesis of rheumatoid arthritis. General Physiology and Biophysics, 18: 15-20, 1999.
Bauerova, K. et al. 2010. Combined methotrexate and coenzyme Q10 therapy in adjuvant-induced arthritis evaluated using parameters of inflammation and oxidative stress. Acta Biochimica Polonica, 57 (3): 347-354.
Begum, G. et al., 2005. Journal of Sport Nutrition and Exercise Metabolism. 15: 493-514.
Boldyrev, A A., et al., 2013. Physiology and Pathophysiology of Carnosine. https://doi.org/10.1152/physrev.00039.2012.
Campo G M, et al. 2006. Antioxidant activity of chondroitin sulfate. Adv. in Pharmacol. 53:417-431.
Cevc, G. and Richardsen, H. 1993. Lipid vesicles and membrane fusion. Advanced Drug Delivery Reviews. 38 (3): 207-232.
Cevc, G. 1993. Rational design of new product candidates: the next generation of highly deformable bilayer vesicles for noninvasive, targeted therapy. Journal of Controlled Release. 160 (2): 135-146.
Chandrasekaran, N. C. et al. 2014. Effects of magnesium deficiency—More than skin deep. Exp. Bio and Med. 239:1280-1291.
Chuang, C. Y. et al. 2014. Oxidation and modification of extracellular matrix and its role in disease. Free Radical Research. 48 (9): 970-989.
Daghestani, H. N. & Kraus, V. B. 2015. Osteoarthritis Cartilage. 23(11):1890-1896.
Dissette V, et al. 2018. Evaluation of the Transepidermal Penetration of a Carnosine Complex in Gel Formulation by 3D Skin Models. Cosmetics. 5(4). 67.
Drafi, F. et al. 2010. Carnosine inhibits degradation of hyaluronan induced by free radical processes in vitro and improves the redox imbalance in adjuvant arthritis in vivo. Neuro Endocrinology Letters. 31(2): 96-100.
Forman H J, et al. 2008. The chemistry of cell signaling by reactive oxygen and nitrogen species and 4-hydroxynonenal. Arch Biochem Biophys. 2008; 477:183-195.
Jukic I, et al. 2021. Carnosine, Small but Mighty—Prospect of Use as Functional Ingredient for Functional Food Formulation. Antioxidants (Basel). 10(7): 1037.
Monfort, J. et al. 2008. Biochemical basis of the effect of chondroitin sulphate on osteoarthritis articular tissues. Annals of the Rheumatic Diseases. 67 (6): 735-40.
Nakamura H, et al. 1997. Redox regulation of cellular activation. Annual Review of Immunology. 15:351-369.
Lee, J. et al. 2018. L-carnosine induces apoptosis/cell cycle arrest via suppression of NF-κB/STAT1 pathway in HCT116 colorectal cancer cells. In Vitro Cell Dev Biol Anim. 54(7):505-512.
Ponist, S. et al. 2016. Effect of Carnosine in Experimental Arthritis and on Primary Culture Chondrocytes. Oxidative Medicine and Cellular Longevity Volume 2016, Article ID 8470589.
Sugimoto M A, et al. 2016. Resolution of inflammation: what controls its onset? Front. Immunol. 7: 160.
Vallieres, M. and du Souich, P. 2010. Modulation of inflammation by chondroitin sulfate. Osteoarthritis Cartilage. 18 Suppl 1: S1-6.
Volpi, n. 2011. Anti-inflammatory activity of chondroitin sulphate: new functions from an old natural macromolecule. Inflammopharmacology 19(6):299-306
Zhang, W. et al., 2015. Mechanistic insights into cellular immunity of chondroitin sulfate A and its zwitterionic N-deacetylated derivatives. Carbohydr. Polym. 123:331-8.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A topical composition comprising L-carnosine, chondroitin 4 sulfate, and a topical vehicle, wherein a combined amount of L-carnosine and chondroitin 4 sulfate ranges from about 0.5-15 wt % of the topical composition.

2. The topical composition of claim 1, wherein the combined amount of L-carnosine and chondroitin 4 sulfate ranges from about 5-10 wt % of the topical composition.

3. The topical composition of claim 1, wherein a weight ratio of L-carnosine to chondroitin 4 sulfate ranges from about 1:1 to about 10:1.

4. The topical composition of claim 3, wherein the weight ratio of L-carnosine to chondroitin 4 sulfate ranges from about 2:1 to about 5:1.

5. The topical composition of claim 1, wherein the chondroitin 4 sulfate is in a form of a liposomal chondroitin 4 sulfate.

6. The topical composition of claim 1, wherein the combination of L-carnosine and chondroitin 4 sulfate achieves a synergistic antioxidant and/or anti-inflammatory effect.

7. The topical composition of claim 1, further comprising a magnesium salt, wherein a combined amount of magnesium and L-carnosine is about 5-10% by weight of the topical composition.

8. The topical composition of claim 7, wherein a molar ratio of magnesium to L-carnosine in the topical composition is about 1:1.

9. The topical composition of claim 1, further comprising one or more ingredients selected from a further anti-inflammatory agent, a preservative, a gel forming agent, a moisturizing agent, a carrier, a surfactant, a chelating agent, and an emollient.

10. The topical composition of claim 1, further comprising one or more ingredients selected from aloe vera, phenoxyethanol, tetrasodium glutamate diacetate, and hydroxyethyl cellulose.

11. The topical composition of claim 1, wherein the topical vehicle is water.

12. The topical composition of claim 1, in the form of a cream, gel, a solution, a lotion, a paste, an ointment, a patch with or without a reservoir, a spray, or a foam.

13. The topical composition of claim 1, in the form of a gel.

14. The topical composition of claim 1, comprising the following ingredients:
1) aloe vera, in an amount of about 0.5-5% by weight of the topical composition;
2) a magnesium salt, in an amount of about 0.5-10% by weight of the topical composition;
3) phenoxyethanol, in an amount of about 0.5-5% by weight of the topical composition;
4) tetrasodium glutamate diacetate, in an amount of about 0.1-5% by weight of the topical composition;
5) L-carnosine, in an amount of about 0.5-10% by weight of the topical composition;
6) hydroxyethyl cellulose, in an amount of about 0.5-5% by weight of the topical composition;
7) liposomal chondroitin 4 sulfate, in an amount of about 0.1-5% by weight of the topical composition; and
8) the topical vehicle.

15. The topical composition of claim 14, characterized by one or more of the following:
1) the aloe vera is in an amount of about 2-4% by weight of the topical composition;
2) the magnesium salt is a magnesium sulfate, which is in an amount of about 1-3%, about 4-6%, or about 5-10% by weight of the topical composition;
3) the phenoxyethanol is in an amount of about 1-3% by weight of the topical composition;
4) the tetrasodium glutamate diacetate is in an amount of about 0.1-1% or about 1-3% by weight of the topical composition;
5) the L-carnosine is in an amount of about 1-4%, about 1.5-2.5%, about 4-6%, or about 5-10% by weight of the topical composition;
6) the hydroxyethyl cellulose is in an amount of about 1-3% by weight of the topical composition;
7) the liposomal chondroitin 4 sulfate is in an amount of about 0.5-2% or about 1-3% by weight of the topical composition; and
8) the topical vehicle is water.

16. The topical composition of claim 14, characterized by one or more of the following:
1) the aloe vera is in an amount of about 3.1% by weight of the topical composition;
2) the magnesium salt is a magnesium sulfate, which is in an amount of about 3% or about 5% by weight of the topical composition;
3) the phenoxyethanol is in an amount of about 1.7% by weight of the topical composition;
4) the tetrasodium glutamate diacetate is in an amount of about 0.5% or about 1.7% by weight of the topical composition;
5) the L-carnosine is in an amount of about 3%, or about 5% by weight of the topical composition;
6) the hydroxyethyl cellulose is in an amount of about 2% or about 2.5% by weight of the topical composition;
7) the liposomal chondroitin 4 sulfate is in an amount of about 1.2% by weight of the topical composition; and
8) the topical vehicle is water.

17. The topical composition of claim 14, wherein a combined amount of magnesium and L-carnosine ranges from about 5-10% by weight of the topical composition.

18. The topical composition of claim 14, comprising, for every 32 oz of the composition, about 1 oz aloe vera (liquid form), about 48.05 grams magnesium sulfate heptahydrate, about 15 grams of phenoxyethanol, about 15 grams of tetrasodium glutamate diacetate, about 45 grams of L-carnosine, about 23 grams of hydroxyethyl cellulose, about 18 grams of liposomal chondroitin 4 sulfate, and water Q.S.

19. A method of preparing a topical composition comprising L-carnosine, chondroitin 4 sulfate, a topical vehicle, and optional one or more other ingredients, the method comprising mixing the L-carnosine and chondroitin 4 sulfate, and optional one or more other ingredients, in the topical vehicle to form a homogenous mixture, wherein a combined amount of L-carnosine and chondroitin 4 sulfate ranges from about 5-15 wt %, preferably, about 5-10 wt % of the topical composition, preferably, the combination of L-carnosine and chondroitin 4 sulfate achieves a synergistic antioxidant and/or anti-inflammatory effect.

20. A method of treating inflammation or a disease associated with an inflammatory and/or autoimmune reaction, the method comprising topically applying an effective amount of the topical composition of claim 1 to a subject in need thereof.
